(19) United States
(12) Patent Application Publication
(10) Pub. No.: US 2018/0110821 A1
(43) Pub. Date: Apr. 26, 2018

(54) ANTI-FATIGUE FOOD COMPOSITION AND ANTI-FATIGUE AGENT

(71) Applicant: MOMOYA CO., LTD., Tokyo (JP)

(72) Inventors: Kenji Hashiguchi, Saitama (JP); Aya Nakamura, Saitama (JP); Yuko Nagata, Saitama (JP); Kyoko Yamasaki, Saitama (JP); Hiroharu Takashige, Saitama (JP); Masataka Saito, Saitama (JP)

(73) Assignee: MOMOYA CO., LTD., Tokyo (JP)

(21) Appl. No.: 15/790,476

(22) Filed: Oct. 23, 2017

(30) Foreign Application Priority Data
Oct. 24, 2016 (JP) 2016-207901
Oct. 18, 2017 (JP) 2016-202109

(51) Int. Cl. A61K 36/8962; A61P 43/00; A23L 33/105

(52) U.S. Cl. CPC A61K 36/8962; A23V 2002/00; A23L 33/105; A61P 43/00

(57) ABSTRACT

Problems

To provide an anti-fatigue food composition exerting an anti-fatigue effect in the form of food to be ingested easily.

Means for solving the problems

An anti-fatigue food composition containing an aged garlic extract aged by heating.

### BACKGROUND OF THE INVENTION

### Field of the Invention

The present invention relates to an anti-fatigue food composition and anti-fatigue agent, and more specifically, to an anti-fatigue food composition and anti-fatigue agent containing an aged garlic extract aged by heating as an active ingredient.

### Background Art

In recent years, great changes in labor circumstances and the living environment by rapid changes in social structure and so on contribute to a tremendous increase in people who feel physical and mental fatigue. A study conducted by the Fatigue Surveillance Test group in the former Ministry of Health and Welfare in 1999 showed that approximately 60% of the population felt fatigue. The presence of chronic fatigue syndrome, which affects people who have been in good health but are suddenly attacked by an unidentified intense lassitude resulting in being incapable of enjoying a healthy social life, has become clear. So the elucidation of the etiology and pathology and development of diagnosis methods and modalities of therapy have followed. From these facts, it is thought that ingesting food in the daily diet that can prevent and alleviate the feeling of fatigue would be beneficial for many people to maintain or improve their health.

It is believed that garlic has been consumed since the time of ancient Egypt, and nowadays it is eaten as a spice due to its characteristic taste and smell, and furthermore, as a food with nutritional value and an appetite stimulating effect. When cells of garlic are destroyed by processes such as cutting, crashing and rubbing, alliin contained in garlic is converted into allicin, which has a characteristic smell and is a pungent component of garlic, by the action of alliinase. Therefore, when raw garlic is ingested, the smell of sulfur compounds such as allicin remains in the mouth and stomach, which makes people feel unpleasantness. Allicin in garlic changes to derivatives such as sulfides by cooking, but these derivatives also contain many ingredients with a strong smell, so the odor after the ingestion frequently lasts for a long time. Also, ingestion of a large quantity of raw garlic may cause a gastrointestinal disorder by the strong stimulation of allicin. The taste and smell of garlic are preferable when eating, but many people avoid ingesting it because of the problems of its odor after eating.

Aged (black) garlic, with the reduced characteristic smell of garlic and without the pungent taste, is produced by inactivating alliinase through the heat-aging of the raw garlic over a long period of time. In the manufacturing process of the aged garlic, polysaccharides such as fructan change to monosaccharides such as fructose. This enhances sweetness by adding a prune-like taste and allowing the aged garlic to be ingested without cooking, so there is an increase in the people who consume it in recent years. Because the heat-aging process remarkably increases functional substances such as S-allylcysteine (SAC) and polyphenols that are not abundant in raw garlic, new features not present in raw garlic are expected.

Aged garlic is usually aged while maintaining a temperature of around 70° C. for about one month, with a great deal of time and care, so it becomes expensive (Patent document 1).

To solve this problem, an aged garlic extract has been reported which is prepared by aging for a short period of time and contains more S-allylcysteine, expected for its functionality, than general aged garlic (Patent document 2).

In general, fatigue in a broad sense is defined as distinct discomfort and lessening of physical or mental performance, that are seen when physical burdens such as exercise or mental stresses such as deskwork are continuously experienced. Therefore, an anti-fatigue food would mean a food that alleviates distinct discomfort and lessening of physical or mental performance that appear when physical or mental burdens are experienced.

In the technical field of the art, a major cause of fatigue is known to be fatigue of the autonomic nerve center in the brain. Also, recent studies have found that the direct cause of fatigue is oxidative stress due to active oxygen species and have clarified that reducing oxidative stress by an antioxidant substance having an antioxidative effect alleviates fatigue. Various substances such as vitamins, polyphenols, carotenoids are known as antioxidative substances. However, only part of them exerts anti-fatigue effect in vivo because the mechanisms of antioxidation of these substances vary and their antioxidative actions are found to be exerted at different time points in vivo. Especially, among them only a few substances can directly contribute to recovery from fatigue in the brain and can pass through the blood-brain barrier.

Regarding anti-fatigue agents containing garlic, in the past there have been prior arts showing an anti-fatigue effect in conjunction with ornithine (Patent document 3), in conjunction with a Chinese chives extract (Patent document 4), and in conjunction with imidazole peptide composed of carnosine, anserine, valerin and salts thereof, or salts thereof (Patent document 5), and by a synergetic effect with a plurality of other functional materials (Patent document 6). Regarding the aged garlic, there was only a prior art reporting that the aged garlic exerted an anti-fatigue effect when used black garlic powder in conjunction with ginseng (Patent document 7). However, the anti-fatigue food composition of this black garlic necessitates to be ingested multiple times a day, therefore, ingesting itself might become a burden and this necessarily was a possible cause of missing ingestion. Regarding the evaluation of the anti-fatigue effect, it was unclear whether long-term ingestion can exert anti-fatigue effect or not because the evaluation was carried out in only two days, a short period for ingestion.

### PRIOR ART DOCUMENT

### Patent Document

Patent Document 1: Japanese Patent No. 4080507

Patent Document 2: Japanese Patent No. 5968729

Patent Document 3: Japanese Patent Application No. 2007-538778

Patent Document 4: Japanese Unexamined Patent Application Publication No. 2008-074792

Patent Document 5: Japanese Unexamined Patent Application Publication No. 2016-008202

Patent Document 6: Japanese Unexamined Patent Application Publication No. 2001-114695

Patent Document 7: Japanese Patent No. 5601747

### SUMMARY OF THE INVENTION

Under the circumstances described above, an anti-fatigue food has been demanded that it has a sustainable effect when taken for a long period, evaluation of the effect can be carried out objectively, and furthermore its ingestion does not cause a burden.

The inventors found that a food composition of an aged garlic extract containing abundant S-allylcysteine with high functionality prepared in a paste form for easy ingestion suppressed accumulation of the feeling of fatigue in daily life, resulting in the improvement of work efficiency. The present invention is based on this finding.

Therefore, an object of the present invention is to provide an anti-fatigue food composition exhibiting an anti-fatigue effect in everyday life in a food form that can be easily ingested.

The following inventions are provided according to the present invention.

The first invention is an anti-fatigue agent containing an aged garlic extract aged by heating.

The second invention is an anti-fatigue agent according to the first invention, used for suppressing of accumulation of the feeling of fatigue in daily life.

The third invention is an anti-fatigue agent according to the first invention, used for obtaining at least one efficacy selected from the group consisting of the followings;

(1) efficacy of improving in working efficiency;

(2) efficacy of suppressing of lessening of the feeling of relaxation;

(3) efficacy of suppressing of accumulation of the feeling of stress;

(4) efficacy of improving in ability to concentrate;

(5) efficacy of suppressing of lessening of feeling of wakefulness;

(6) efficacy of improving of feeling of wakefulness;

(7) efficacy of suppressing of lessening of the state of liveliness;

(8) efficacy of improving in the feeling of sound sleep;

(9) efficacy of alleviating the feeling of fatigue at the time of awakening;

(10) efficacy of maintaining of balance of the autonomic nerves;

(11) efficacy of improving in the balance of the autonomic nerves.

The forth invention is an anti-fatigue food containing an anti-fatigue agent according to any one of the first invention to the third invention.

The fifth invention is an anti-fatigue food according to the forth invention, wherein the food is a paste containing an aged garlic extract aged by heating, brown sugar, black vinegar, and sugar.

The sixth invention is an anti-fatigue food containing an aged garlic extract aged by heating.

The seventh invention is an anti-fatigue composition according to the sixth invention, used for suppressing of accumulation of the feeling of fatigue in everyday life.

The eighth invention is the anti-fatigue composition according to the seventh invention, used for obtaining at least one of efficacy selected from the group consisting of the followings;
(1) efficacy of improving in working efficiency;

(2) efficacy of suppressing of lessening of the feeling of relaxation;

(3) efficacy of suppressing of accumulation of the feeling of stress;

(4) efficacy of improving in ability to concentrate;

(5) efficacy of suppressing of lessening of feeling of wakefulness;

(6) efficacy of improving of feeling of wakefulness;

(7) efficacy of suppressing of lessening of the state of liveliness;

(8) efficacy of improving in the feeling of sound sleep;

(9) efficacy of alleviating the feeling of fatigue at the time of awakening;

(10) efficacy of maintaining of balance of the autonomic nerves;

(11) efficacy of improving in the balance of the autonomic nerves.

The ninth invention is the anti-fatigue composition according to the seventh invention or eighth invention, wherein the composition is a food composition.

The tenth invention is the anti-fatigue composition according to the ninth invention, wherein the composition is a paste containing an aged garlic extract aged by heating, brown sugar, black vinegar, and sugar.

According to an anti-fatigue agent, an anti-fatigue food and drink product and an anti-fatigue composition of the present invention, it becomes possible to suppress accumulation of the feeling of fatigue in daily life and to improve work efficiency by suppressing the feeling of fatigue. Particularly, the present invention is advantageous in terms of producing effects such as suppression of accumulation of the feeling of fatigue in everyday life, improvement in work efficiency, suppression of lessening of the feeling of relaxation, suppression of accumulation of the feeling of stress, improvement in ability to concentrate, suppression of lessening of the feeling of wakefulness, improvement in the feeling of wakefulness, suppression of lessening of the state of liveliness, improvement in the feeling of sound sleep, alleviation of the feeling of fatigue at the time of awakening, maintenance of the balance of the autonomic nerves and improvement in the balance of the autonomic nerves.

### BRIEF DESCRIPTION OF THE DRAWINGS

FIG. 1 shows a temporal change in the feeling of fatigue (VAS: Visual Analog Scale) of a test group and a placebo group during the study period of Example 1.

FIG. 2 shows a temporal change in the feeling of fatigue (POMS: Profile of Mood States) of the placebo group during the study period of Example 1.

FIG. 3 shows a temporal change in fatigue (POMS) of the test group during the study period of Example 1.

FIG. 4 shows a temporal change in the number of work by the Kraepelin test in the test group and the placebo group during the study period of Example 1.

FIG. 5 shows a temporal change in the feeling of wakefulness (SACL: Stress Arousal Check List) of the placebo group during the study period of Example 1.

FIG. 6 shows a temporal change in the feeling of wakefulness of the test group (SACL) during the study period of Example 1.

FIG. 7 shows a temporal change in the feeling of relaxation (VAS) of the test group and the placebo group during the study period of Example 1.

FIG. 8 shows a temporal change in the feeling of stress (SACL) of the placebo group during the study period of Example 1.

FIG. 9 shows a temporal change in the feeling of stress (SACL) of the test group during the study period of Example 1.

FIG. 10 shows a temporal change in ability to concentrate (VAS) of the test group and the placebo group during the study period of Example 1.

FIG. 11 shows a temporal change in the feeling of liveliness among the items belonging to the feeling of relaxation (SACL) of a test group and a placebo group during the study period of Example 1.

FIG. 12 shows a temporal change in the feeling of fatigue (VAS: Visual Analog Scale) of a test group and a placebo group during the study period of Example 2.

FIG. 13 shows a temporal change in the feeling of mental fatigue at the time of awakening (five-grade evaluation in a diary) of a test group and a placebo group during the study period of Example 2.

FIG. 14 shows a temporal change in the feeling of physical fatigue at the time of awakening (five-grade evaluation in a diary) of a test group and a placebo group during the study period of Example 2.

FIG. 15 shows a temporal change in sum of the feeling of mental fatigue and physical fatigue at the time of awakening (five-grade evaluation in a diary) of a test group and a placebo group during the study period of Example 2.

FIG. 16 shows a temporal change in the feeling of sound sleep (five-grade evaluation in a diary) of a test group and a placebo group during the study period of Example 2.

FIG. 17 shows a temporal change in the feeling of wakefulness of the test group and a placebo group (SACL) during the study period of Example 2.

FIG. 18 shows a change in the balance of the sympathetic nerve and parasympathetic nerve of a placebo group before and after the study period of Example 2.

FIG. 19 shows a change in the balance of the sympathetic nerve and parasympathetic nerve of a test group before and after the study period of Example 2.

FIG. 20 shows content ratio of garlic derived component in the aged garlic extract aged by heating and commercial samples A and B.

### SPECIFIC EXPLANATION OF INVENTION

An anti-fatigue agent, an anti-fatigue food and drink product and an anti-fatigue composition in the present invention contain S-allylcysteine as an active ingredient. Furthermore, according to a preferred embodiment in the present invention, an anti-fatigue agent, an anti-fatigue food and drink product and an anti-fatigue composition in the present invention contain aged garlic extract as the source of supply of S-allylcysteine.

### Aged Garlic Extract

In the present invention, “an aged garlic extract” or “a black garlic extract” means an extract obtained by processing aged garlic produced by a heat-aging process of garlic. The aging process of garlic allows the increase in the content of high functional ingredients such as S-allylcysteine or polyphenols while reducing the characteristic smell and a sharp taste of garlic.

For raw materials of “the aged garlic extract” in the present invention, dry garlic, raw garlic, salted garlic and the like can be used. For these raw garlic materials, usually garlic with its skin peeled off is used as it is, but appropriately cut or fragmented garlic may also be used if required.

According to a preferred embodiment in the present invention, “an aged garlic extract” is produced by a method described in the Japanese Unexamined Patent Application Publication No. 2014-45693, and the aged garlic extract produced in such a method is granted as enriched with S-allylcysteine, cycloalliin, polyphenols and the like that are highly physiologically active substances by heat-treating a raw garlic material for a short period.

Specifically, when dry garlic raw materials are used, an extract (in the form of solid and/or liquid) obtained in principle by adding water to dry garlic raw materials and extracting them can be aged at a temperature of 90° C. or more. The proportion of water used relative to the dry garlic raw materials is usually around 2 to 12 parts by weight, and more preferably 3 to 4 parts by weight relative to 1 part by weight of garlic with the skin peeled off. Also, the temperature of the water used is usually around 5 to 70° C., and preferably 20 to 40° C. The extraction time is usually 20 to 60 minutes, and preferably 30 to 40 minutes. By these processes, a garlic extract (including the solid part of garlic) is obtained.

When dry garlic raw materials are used, regarding an extract (including the solid part of garlic) obtained after the extraction processing, a liquid part of the extraction obtained by filtering the solid part of garlic with a filter cloth or the like can be usually used for a next heat-aging step. In this case, the extract including the solid part of the garlic can be used. However, if the solid part of garlic is removed at this stage, the subsequent process will be more effective because the intended highly physiologically active substance is sufficiently extracted in this extract liquid. For such an extract of garlic, the form of a garlic immersion liquid produced in a soaking step for dry garlic raw materials can be used because it is obtained under a similar condition to the above. It is desirable because the immersion liquid can be also utilized.

The garlic extract obtained by extraction processing (an extract in a preferable embodiment) can be subsequently subjected to heat-aging at a temperature of 90° C. or more. The temperature for heat-aging can be set approximately at 90° C. to 135° C., preferably 90° C. to 130° C., and more preferably 100° C. to 130° C. When heated at 100° C. or more, the heat treatment is carried out under the conditions including ordinary pressure or more using a pressurized vessel (e.g., an autoclave, a pressure kneader). The heat-aging period is usually two hours to seven days, preferably two hours to two days (heating temperature of 130 to 100° C.). The heat-aging step is carried out either in an open condition or a sealed condition, but the sealed-system using airtight containers (e.g., a kneader, an autoclave) is preferable in view of gustatory sensation of the aged garlic extract after aging, increase of the amount of physiologically active substances, workability and the like. As a guide, completion of the aging step is determined by the aging period mentioned above, pH of the extract, sensory evaluation, and the like. Aged garlic extract in the form of liquid or paste is obtained by such a heat-aging process. The pH of this aged garlic extract is usually 3.5 to 5.0 and preferably 4.0 to 4.5, and the reading on the sugar refractometer (Brix) is 5 to 50, and preferably 10 to 30.

In the present invention, garlic extract obtained in the way described above can be used as an aged garlic extract, but it is preferable that a concentration procedure be carried out after the heat-aging step in order to further increase the concentration of the desired physiologically active substances. The concentration procedure is carried out generally using steam in an open condition or using a vacuum concentration machine (usually under the condition of 50 to 700 Torr of reduced pressure) up to the desired degree of the reading on the sugar refractometer (Brix) at 50 to 70° C., generally around 45 to 55 (pH 3.5 to 5.0) and preferably 48 to 52 (pH 4.0 to 4.5), and the produced solid part is removed with a filter cloth and the like. Degree of concentration can be obtained by approximately adjusting temperature, time, Brix, degree of reduced pressure and the like. Concentrated aged garlic extract is thus obtained by the method mentioned above. Also, although the Brix of the garlic extract under the thermal extraction is usually approximately 5 to 20 in the method mentioned above, aged garlic extract containing high concentrations of components can be obtained without performing concentration procedure by using, in the procedure of heat-aging step, an extract with high degree of Brix (e.g., Brix 20 to 50) obtained by reducing the proportion of water to be added with respect to garlic or by concentration under reduced pressure.

The concentration of S-allylcysteine in the food and drink product of the present invention is 0.3 mg/100 g to 1000 mg/100 g, preferably 0.5 mg/100 g to 1000 mg/100 g, more preferably 1 mg/100 g to 500 mg/100 g, and further preferably 2 mg/100 g to 500 mg/100 g.

The amount of daily ingestion of S-allylcysteine, which is contained in the food and drink product of the present invention, is 0.1 mg to 300 mg, preferably 0.1 mg to 100 mg, more preferably 0.5 mg to 50 mg, and further preferably 0.5 mg to 5 mg.

### Measurement Method of S-Allylcysteine

The concentration of S-allylcysteine can be measured, for example, as follows. At first, to 10 g of a sample is added 490 g of distilled water and the mixture is stirred well. This diluted solution can be analyzed by liquid chromatography using AccQ-Tag amino acid analysis (Waters). Preferably, the analysis can be conducted by UPLC (registered trademark) (Ultra High Performance Liquid Chromatography), and UPLC conditions can be set as follows.

### (UPLC Analysis Conditions)

Device: UPLC system (Nihon Waters K. K.)
Column: AccQ-Tag Ultra C18 (1.7 μm, 2.1 mm×100 mm)
Mobile phase solution A: AccQ-Tag Ultra eluent A concentrated (Waters)
Mobile phase solution B: solution A obtained by diluting solution D with Milli-Q water by a factor of 10
Mobile phase solution C: Milli-Q water
Mobile phase solution D: Acetonitrile/formic acid=97/3
Gradient: Set as in the following Table 1.

TABLE 1

| Time | Initial | | | | | | | | | |
|---|---|---|---|---|---|---|---|---|---|---|
| (minute) | value | 0.29 | 5.49 | 7.1 | 7.3 | 7.69 | 7.99 | 8.59 | 8.68 | 10.20 |
| Solution A | 10.0 | 9.9 | 9.0 | 8.0 | 8.0 | 7.8 | 4.0 | 4.0 | 10.0 | 10.0 |
| (%) | | | | | | | | | | |
| Solution B | 0.0 | 0.0 | 80.0 | 15.6 | 15.6 | 0.0 | 0.0 | 0.0 | 0.0 | 0.0 |
| (%) | | | | | | | | | | |
| Solution C | 90.0 | 90.1 | 11.0 | 57.9 | 57.9 | 70.9 | 36.3 | 36.3 | 90.0 | 90.0 |
| (%) | | | | | | | | | | |
| Solution D | 0.0 | 0.0 | 0.0 | 18.5 | 18.5 | 21.3 | 59.7 | 59.7 | 0.0 | 0.0 |
| (%) | | | | | | | | | | |

Column oven temperature: 43° C.
Sample room temperature: 20° C.
Flow rate: 0.7 mL/min
Detection wavelength: Fluorescence Ex (excitation wavelength) 266 nm, Em (fluorescence wavelength) 473 nm

In the present specification, it is demonstrated that effect of suppressing accumulation of the feeling of fatigue, improvement in work efficiency, effect of suppressing lessening of the feeling of relaxation, effect of suppressing lessening of the feeling of wakefulness, effect of suppressing accumulation of the feeling of stress, effect of improving ability to concentrate are obtained by ingestion of the aged garlic extract. Thus, an anti-fatigue agent, an anti-fatigue food and drink product and an anti-fatigue composition of the present invention are used preferably for the purpose of suppressing accumulation of the feeling of fatigue.

“The feeling of fatigue” in the present invention means a sense of recognizing that fatigue is present. Herein, “fatigue” refers to “decrease of ability of activities caused by overloading of the mind and body”. Also, “anti-fatigue food,” refers to “a food that alleviates lessening of the physical or mental performance that appears when a physical or mental load is imposed”.

“The feeling of relaxation” in the present invention refers to a sense of recognizing relaxation. Herein, “relaxation” refers to “relaxing, ease up, relief strain” (Kojien sixth edition, Japanese dictionary published by Iwanami).

“The feeling of wakefulness” in the present invention refers to a sense of recognizing being wakeful. Herein, “wakefulness” refers to “getting awake, waking up” (Kojien sixth edition, Japanese dictionary published by Iwanami) “The state of liveliness” refers to “lively”, which is one of the question items about the feeling of wakefulness in the Stress Arousal Check List (SACL) in Japanese-translated version, and classified in “General Activation” (Stress, Stress Factors, and Self-report Measures; Clarification of Power, a New Factore, Particia J. Wheeler). And “General Activation” refers to one of the four dimensions of wakefulness in psychology, “tension (High Activation)”, “calmness (General Deactivation)”, “energy (General Activation)”, and “tiredness (Deactivation-Sleep) (Study Proceedings of Kawamura-gakuen women's university, Vol. 6, No. 1, Pages 111 to 123, 1995, Yu Tanaka).

“The feeling of stress” in the present invention refers to a sense of recognizing having stress, and “stress” refers to functional changes brought about in the mind and body when various kinds of external stimulations work as a burden (Kojien sixth edition, Japanese dictionary published by Iwanami).

Also, “ability to concentrate” in the present invention refers to the competence of sustaining consciousness and attention toward certain things (Kojien sixth edition, Japanese dictionary published by Iwanami).

### Application of the Anti-Fatigue Composition of the Present Invention as a Pharmaceutical Product

When the anti-fatigue agent and anti-fatigue composition of the present invention are provided as a pharmaceutical product, such a pharmaceutical product can be produced by mixing the aged garlic extract aged by heating of the present invention with pharmaceutically acceptable carriers.

The anti-fatigue agent and anti-fatigue composition of the present invention can be administered orally or parenterally as an active ingredient, preferably administered orally. Oral formulations include granules, powders, tablets (including sugar-coated tablets), pills, capsules, syrup, emulsions and suspensions. Parenteral formulations include parenteral injections (e.g., subcutaneous injections, intravenous injections, intramuscular injections and intraperitoneal injections), drops, drugs for external use (e.g., transnasal administrative drugs, transdermal formulations and ointment), suppositories (e.g., rectal suppositories, vaginal suppositories). These formulations can be prepared using pharmaceutically acceptable carriers by techniques that are conventionally performed in the field of the art. Pharmaceutically acceptable carriers include excipients, binders, diluents, additives, fragrance, buffers, thickening agents, coloring agents, stabilizers, emulsifiers, dispersing agents, suspending agents, antiseptic agents and the like. For example, magnesium carbonate, magnesium stearate, talc, sugar, lactose, pectin, dextrin, starch, gelatin, tragacanth, methylcellulose, sodium carboxymethyl cellulose, low melting point wax, and cacao butter can be used as a carrier.

Formulations, for example, can be produced as follows.

The oral formulation can be produced by adding, for example, excipients (e.g., lactose, sucrose, starch, mannitol), disintegrating agents (e.g., calcium carbonate, carboxymethyl cellulose calcium), binders (e.g., alpha starch, Arabian gum, carboxymethyl cellulose, polyvinyl pyrrolidone, hydroxypropyl cellulose) or lubricants (e.g., talc, magnesium stearate, polyethylene glycol 6000) to an active ingredient, followed by compressing and molding, and subsequently coating by known methods for the purpose of masking taste, acquiring enteric property or sustained release when needed. For a coating agent, for example, ethyl cellulose, hydroxymethyl cellulose, polyoxyethylene glycol, cellulose acetate phthalate, hydroxypropylmethyl cellulose phthalate and EUDRAGIT (methacrylic acid-acrylic acid copolymer, Rohm Corporation, Germany) and the like can be used.

Parenteral injections can be produced by dissolving, suspending or emulsifying an active ingredient in an aqueous solvent (e.g., distilled water, physiological saline solution, a Ringer's solution and the like) or a lipophilic solvent (e.g., vegetable oil such as olive oil, sesame oil, cotton seed oil and corn oil, propylene glycol) with dispersants (e.g., Tween 80 (Atlas Powder Company, USA), HCO60 (Nikko chemicals Co., Ltd.), polyethylene glycol, carboxymethyl cellulose, sodium alginate and the like), preservatives (e.g., methylparaben, propylparaben, benzyl alcohol, chlorobutanol, phenol), tonicity adjusting agents (e.g., sodium chloride, glycerin, sorbitol, glucose, inverted sugar). In this case, if desired, additives such as solubilizing agents (e.g., sodium salicylate, sodium acetate), stabilizers (e.g., human serum albumin) and analgesics (e.g., benzalkonium chloride, procaine hydrochloride) may be added.

Drugs for external use can be produced by converting an active ingredient to a composition in the form of solid, semisolid or liquid. For example, the above solid composition can be produced by using the active ingredient itself or adding and mixing with excipients (for example, lactose, mannitol, starch, microcrystalline cellulose and sucrose), thickeners (e.g., natural gums, cellulose derivative, and acrylic acid polymer) and the like, and pulverizing. The above liquid composition can be produced almost in the same way of the case of the preparation of parenteral injections. For the semisolid composition, aqueous or lipophilic gel or an ointment-like composition is preferable. Also, any of these compositions may include pH modifiers (e.g., carbonic acid, phosphoric acid, citric acid, hydrochloric acid, sodium hydroxide), antiseptic agents (e.g., p-hydroxybenzoic esters, chlorobutanol, benzalkonium chloride). The suppository can be made by arranging an active ingredient to a composition in the form of aqueous or lipophilic solid, semisolid or liquid. Lipophilic bases used for the said composition include glyceride of higher fatty acids (e.g., cacao butter, Witepsols, (Dynamite Nobel Co., Ltd), middle fatty acid (for example, Migliols (Dynamite Nobel Co., Ltd)) or vegetable oil (e.g., sesame oil, soybean oil, cotton seed oil). Aqueous bases include polyethylene glycols and propylene glycol. Also, aqueous gels bases include natural gums, cellulose derivatives, vinyl polymers and an acrylic acid polymers.

### Application of the Anti-Fatigue Composition of the Present Invention as a Food

The anti-fatigue composition of the present invention can also be utilized as a food additive. Therefore, according to another embodiment in the present invention, a food additive containing the same ingredient as the anti-fatigue composition of the present invention is provided. The anti-fatigue composition of the present invention can be also provided as a food composition. According to another embodiment in the present invention, a food composition containing the anti-fatigue composition of the present invention or the same ingredient as the anti-fatigue composition of the present invention is provided.

The aged garlic extract exerts a physiological effect, particularly suppression of accumulation of the feeling of fatigue in everyday life, as mentioned previously. Therefore, the anti-fatigue agent and anti-fatigue composition of the present invention include those are intended to be added to food in the expectation of this physiological effect. Subjects and embodiments of addition can comply with description regarding the food composition of the present invention.

The food composition of the present invention is a food and drink product containing the anti-fatigue composition of the present invention or the same ingredients as the anti-fatigue composition of the present invention at an effective dose. Herein, “containing at an effective dose” means such a dose that the aged garlic extract is ingested within a range as is described below when each food and drink product is ingested at a usual amount for eating and drinking.

When the anti-fatigue composition of the present invention is provided as a food composition, the anti-fatigue composition of the present invention or the same ingredients as the anti-fatigue composition of the present invention can be blended with food as it is. Specifically, the food composition of the present invention may be a food and drink product prepared from the anti-fatigue composition of the present invention as it is; a preparation further blended with various proteins, saccharides, lipids, microelements, vitamins and the like; a preparation in the form of liquid, semiliquid or solid; a preparation in the form of aqueous solution of a potassium salt and sodium salt; or a food composition prepared by adding the anti-fatigue composition to a general food and drink product.

“A food composition” in the present invention is used in referring to a common food (including so-called health food), food for special dietary uses and a food with health claims (including a food for specified health use, a food with nutrient function claims, and a food with function claims).

The food and drink product of the present invention may include other ingredients used for producing a food and drink product containing the aged garlic extract. As these other ingredients, for example, vinegars (e.g., black vinegar, rice vinegar, grain vinegar, fruit vinegar), sugar (e.g., superfine sugar, brown sugar), condiments (e.g., Mirin (sweet cooking rice wine), salt), sweeteners (e.g., glucose, fructose, oligosaccharide, isomerized sugar syrup, sugar alcohol, high-intensity sweeteners), acidifiers (e.g., citric acid, malic acid, tartaric acid, lactic acid, phosphoric acid, phytic acid, itaconic acid, fumaric acid, gluconic acid, adipic acid, acetic acid or salts thereof), pigments, food additives (e.g., preservatives, antioxidizing agents, dietary fibers, emulsifiers) can be appropriately added.

The food and drink product of the present invention is preferably provided as a packed food and drink product. The container used for the food and drink product of the present invention may be one generally used for holding food and drink products, and includes, for example, plastic bottles (e.g., PET bottle, cup), paper containers, bottles, pouch containers.

Beverages provided in the present invention include, for example, water, refreshing beverages, fruit juice beverages, tea beverages, milk beverages, isotonic drinks, nutrition-supplement drinks. Food provided in the present invention includes, for example, bread, noodles, rice products, side dishes, dairy products, seasonings, confectionery.

The food composition of the present invention may further contain additives generally used in the field of the art within a range not to affect the effect of the present invention. Such additives include, for example, sweeteners, acidifiers, fragrance, antioxidizing agents, bittering agents, apple fibers, soybean fibers, meat extracts, black vinegar extracts, gelatin, cornstarch, honey, animal and vegetable oil and fat; proteins such as gluten; amino acids; peptides; monosaccharides such as glucose and fructose; disaccharides such as sucrose; polysaccharides such as dextrose and starch; sugar alcohols such as erythritol, xylitol, sorbitol and mannitol; vitamins such as vitamin C; minerals such as zinc, copper and magnesium; functional materials such as CoQ10, α-lipoic acid, carnitine, capsaicin and polyphenols; fruit juice; and milk or milk constituents. These additives can be used separately or in combination of multiple kinds thereof.

### EXAMPLES

The present invention will be specifically explained according to the following examples, but the present invention is not limited to these examples.

### [Test Contents]

### [Preparation of an Aged Garlic Extract]

To 10 kg of dried garlic was added 40 kg of water, and the mixture was heated and aged at 100° C. for 32 hours. After the mixture was finally concentrated up to Brix 50 (the reading on a sugar refractometer), the solid part was removed by filtration. Other preparation methods were carried out according to the method described in the Japanese Unexamined Patent Application Publication No. 2014-45693 disclosed by the inventors of the present invention. The extract obtained was served as an aged garlic extract.

### [Preparation of an Aged Garlic Extract Paste]

The aged garlic extract, brown sugar syrup, sugar and black vinegar were mixed while heating. The paste obtained was served as a test food.

### [Preparation of a Black Syrup Paste]

Commercially available brown sugar syrup, sugar, black vinegar, liquid sugar, brewed vinegar were mixed while heating, and the paste obtained was served as a placebo food.

The content of the aged garlic extract was 12.5 g in 100 g of the aged garlic extract paste used as the test food. Also, levels of nutritional components of the test food and the placebo food are described in Table 2.

TABLE 2
Value of nutrients in the test food and placebo food (per 100 g)

| | | | | | Carbo- | Saccha- | Dietary | Ash | |
|---|---|---|---|---|---|---|---|---|---|
| Sample | Energy | Water | Protein | Lipid | hydrate | ride | fiber | content | Sodium |
| Name | kcal | % | g | | | | | | mg |
| Test | 278 | 29.3 | 1.4 | 0.1 | 68.0 | 68.0 | 0.0 | 1.3 | 46.2 |
| food | | | | | | | | | |
| Placebo | 268 | 29.5 | 0.6 | 0.0 | 68.7 | 68.7 | 0.0 | 1.2 | 9.6 |
| food | | | | | | | | | |

### [S-Allylcysteine Analysis]

Analysis Method

To 10 g of a sample was added 490 g of distilled water and the mixture was stirred well. S-allylcysteine can be measured using this diluted solution by liquid chromatography using AccQ-Tag method of amino acid analysis (Waters). Preferably, the analysis can be carried out by UPLC (registered trademark) (Ultra High Performance Liquid Chromatography) under the UPLC conditions described as follows.

(UPLC analysis conditions)
Device: UPLC system (Nippon Waters K.K.)
Column: AccQ-TagUltraC18 (1.7 μm, 2.1 mm×100 mm)
Mobile phase solution A: AccQ-Tag Ultra eluant A concentrate (Waters)
Mobile phase solution B: Solution obtained by diluting solution D with
Milli-Q water by a factor of 10
Mobile phase solution C: Milli-Q water
Mobile phase solution D: Acetonitrile/formic acid=97/3
Gradient: Set as in the following Table 3.

TABLE 3

| Time | Initial | | | | | | | | | |
|---|---|---|---|---|---|---|---|---|---|---|
| (minute) | value | 0.29 | 5.49 | 7.1 | 7.3 | 7.69 | 7.99 | 8.59 | 8.68 | 10.20 |
| Solution A | 10.0 | 9.9 | 9.0 | 8.0 | 8.0 | 7.8 | 4.0 | 4.0 | 10.0 | 10.0 |
| (%) | | | | | | | | | | |
| Solution B | 0.0 | 0.0 | 80.0 | 15.6 | 15.6 | 0.0 | 0.0 | 0.0 | 0.0 | 0.0 |
| (%) | | | | | | | | | | |
| Solution C | 90.0 | 90.1 | 11.0 | 57.9 | 57.9 | 70.9 | 36.3 | 36.3 | 90.0 | 90.0 |
| (%) | | | | | | | | | | |
| Solution D | 0.0 | 0.0 | 0.0 | 18.5 | 18.5 | 21.3 | 59.7 | 59.7 | 0.0 | 0.0 |
| (%) | | | | | | | | | | |

Column oven temperature: 43° C.
Sample room temperature: 20° C.
Flow rate: 0.7 mL/min
Detection wavelength: Fluorescence Ex (excitation wavelength) 266 nm, Em (fluorescence wavelength) 473 nm

Analysis Results

According to the results of S-allylcysteine analysis, the content was 17.7 mg/100 g for the test food and 0.0 mg/100 g for the placebo food (Table 4).

TABLE 4
Contents of high functional ingredients per
100 g in the test food and placebo food

| | |
|---|---|
| S-allyl cysteine | Polyphenols |

| | |
|---|---|
| Sample Name | mg |

| | | |
|---|---|---|
| Test food | 17.7 | 373.2 |
| Placebo food | 0.0 | 275.0 |

### Example 1: Ingestion Test 1

### [Design for Human Test]

An ingestion test was carried out by a randomized, double-blind placebo-controlled parallel-group trial. In addition, all the tests of the present application obtained an approval from the Ethical Review Committee at Kagawa Nutrition University and were performed under instructions by experts.

### [Age of Subjects, the Number of Subjects]

The ingestion test was conducted for a total of 49 healthy men and women subjects of 20 or more and less than 65 years of age.

### [Test Period]

A test was carried out for two weeks for a pre-observation period and four weeks for the ingestion period, for a total of 6 weeks. The test period was set from Jun. 5 to Jul. 24, 2015, for a total of 6 weeks. Because the rainy season began in the Kanto Koshin district on June 3, and ended on Jul. 10, 2015, there were mostly rainy days or days with high humidity during the test period. The maximum ambient temperature ranged from 20° C. to 35° C. showing a large temperature difference, and the ambient temperature gradually increased so that people easily felt fatigue during the test period. Also, for students included as subjects, the test period overlapped a period busy with exams or practical trainings, so that they further easily felt fatigue in this season.

### [Measurement Items]

Questionnaire Evaluating Emotions

Using a questionnaire, the subjects were asked to address the following three kinds of parameters.

### (1) VAS (Visual Analogue Scale):

A 10-cm-long line was used as a scale, at both ends on which were marked “feeling extremely” and “feeling not at all”. The evaluation was carried out by the position of markings put on the scale by the subjects to indicate the current state of their feeling with respect to the feeling of fatigue, the feeling of relaxation, and ability to concentrate.

### (2) POMS (Profile of Mood States):

The evaluation of emotions was performed by responding with score from one to five, “Extremely” to “Not at all”, to 30 questions regarding tension/anxiety, depression/being discouraged, anger/aggression, weariness, confusion, and vigor.

### (3) SACL (Stress/Wakefulness Check List):

The evaluation of emotions regarding stress or the feeling of wakefulness was performed by answering using a system of scoring from one to four to 30 questions. Further, among the 30 questions, more detail evaluation was performed about “lively”, one of the items classified in “Positive Arousal”.

A Simple Addition Test (Kraepelin Test):

The work efficiency of the subjects was evaluated by letting the subjects repeatedly add up one-digit numbers and counting their number of work and the number of erroneous answers.

The Body Weight, BMI, Body Fat Percentage, Blood Pressure, Pulse, Body Temperature, and Blood Components

### [Schedule of the Measurement Date]

The measurement was carried out in the following order on the measurement date. Measurement of body weight, measurement of blood pressure, blood sampling->emotional evaluation questionnaire->measurement of body temperature->resting time (20 minutes)->Kraepelin test (15 minutes)->end

### [Grouping]

Each measurement was carried out at a briefing session of the ingestion test and grouping was randomly carried out according to the data obtained. There was no difference between the groups regarding all the items measured in this test. During the study period, a group for ingesting the test food was set as a test group. A group for ingesting the placebo food was set as a placebo group.

### [Test Contents]

Based on the result of grouping, 5 g of the test food or placebo food were ingested once per day for four weeks. Each measurement mentioned above was conducted on the starting date of ingestion, two weeks after and four weeks after the onset of ingestion.

### [Statistical Analysis]

The results were expressed in terms of the mean±standard deviation with respect to fatigue on VAS, simple work efficiency, the feeling of relaxation on VAS and ability to concentrate on VAS, and a parametric test was carried out regarding these parameters. Fatigue scores, stress scores and the feeling of wakefulness scores were expressed as a percentile (90, 75, 50, 25, and 10), and a nonparametric test was carried out regarding these scores.

All the verification was conducted by a two-tailed test at a significance level of 5% (in the Figures, a significance level of 5% was expressed as “*”, and a significance level of 1% was expressed as “**”). In the case where p-values fall in the range of 0.05<p<0.1, the result was evaluated as “having a tendency”, although there was no significance.

### [Results and Discussions]

### (1) Feeling of Fatigue

### Results on VAS

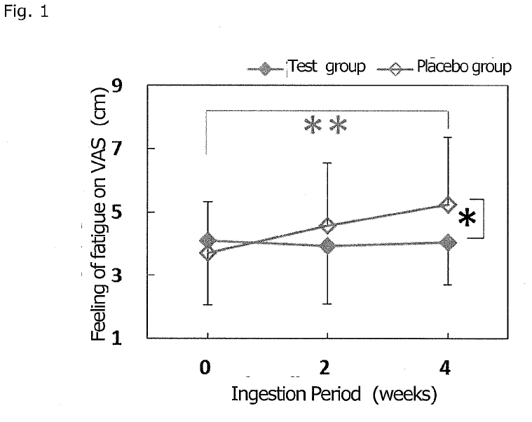

In the placebo group, as the test period overlapped with the period when the feeling of fatigue in the subjects was elevated as mentioned above, a tendency of increasing the feeling of fatigue on VAS was observed (p=0.095) at two weeks after the onset of ingestion compared to the starting date of ingestion, and the feeling of fatigue on VAS increased significantly at four weeks after the onset of ingestion (p=0.006) (FIG. 1). In the test group, on the other hand, no increase in the feeling of fatigue on VAS was found during the test period, and at four weeks after the onset of ingestion, the feeling of fatigue on VAS was significantly lower than that in the placebo group (p=0.03).

### Results of POMS

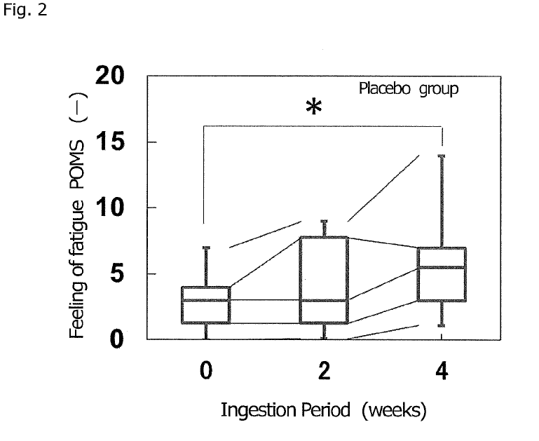

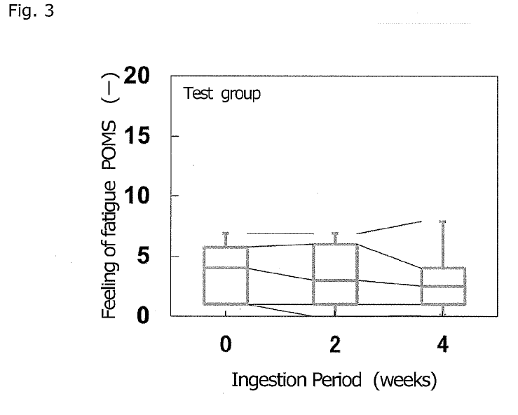

Also, in the placebo group, fatigue scores significantly increased at four weeks after the onset of ingestion compared to those on the starting date of ingestion (p=0.035) (FIG. 2). In the test group, on the other hand, fatigue scores did not increase from the starting date of ingestion to four weeks after the onset of ingestion (FIG. 3).

From these results, the test food was shown to suppress daily accumulation of the feeling of fatigue.

### (2) Simple Work Efficiency

### Results of Kraepelin Test

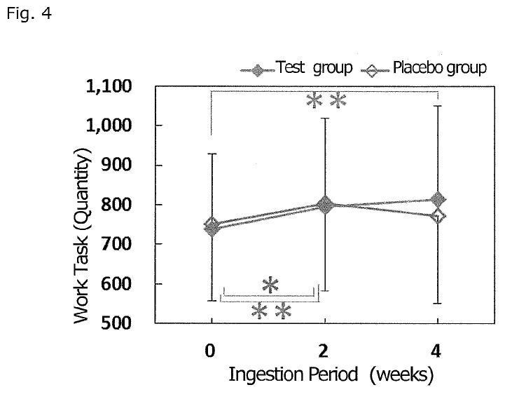

In the placebo group, the number of work significantly increased at two weeks after the onset of ingestion compared to that on the starting date of ingestion (p=0.014), but the number of work at four weeks after the onset of ingestion was similar to that at two weeks after the onset of ingestion (FIG. 4). In the test group, on the other hand, the number of work significantly increased at two weeks after the onset of ingestion (p=0.003) and at four weeks after the onset of ingestion (p=0.003) compared to that on the starting date of ingestion.

From these results, the test food was shown to improve simple work efficiency.

### (3) Feeling of Wakefulness

### Results of SACL

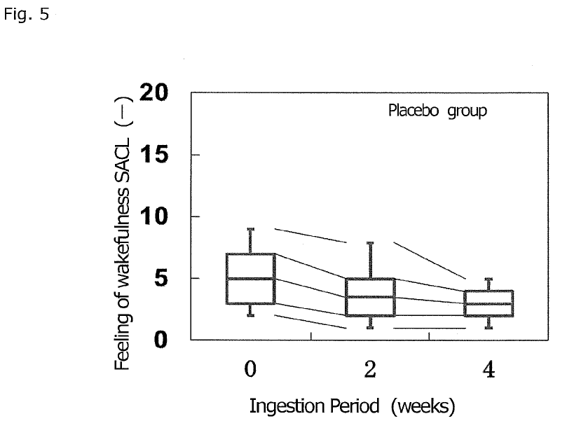

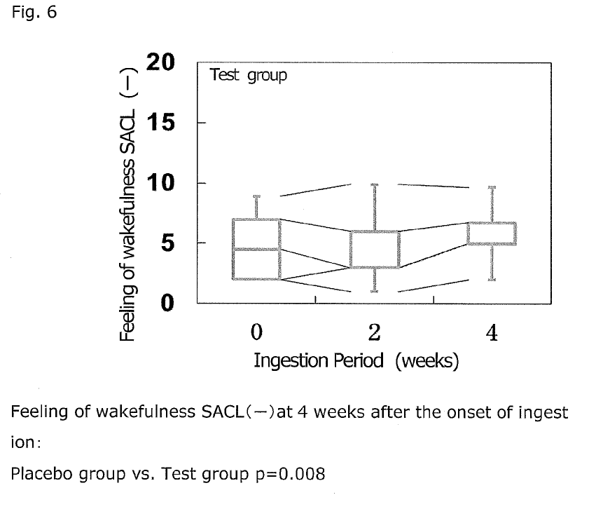

In the placebo group, the scores on the feeling of wakefulness had a tendency to decrease at four weeks after the onset of ingestion compared to those on the starting date of ingestion (p=0.081) (FIG. 5). In the test group, on the other hand, there was no difference in the feeling of wakefulness scores from the starting date to four weeks after the onset of the ingestion, and at four weeks after the onset of ingestion, the feeling of wakefulness scores in the test group were significantly higher than those in the placebo group (p=0.004) (FIG. 6).

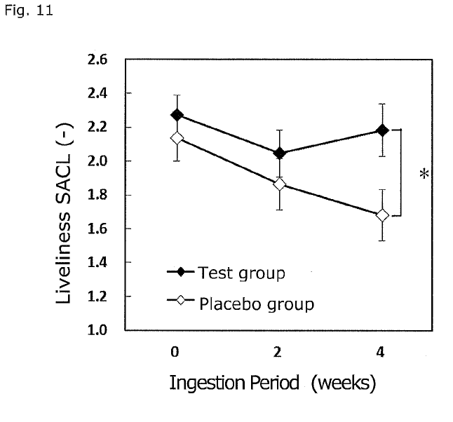

In the result of “lively” among the question items about wakefulness SACL, significant difference can be observed between a test group and a placebo group (p=0.0497) (FIG. 11). In the placebo group, the result showed that the feeling of wakefulness lessened, while in the test group the test food was shown to suppress lessening of the feeling of wakefulness.

### (4) Feeling of Relaxation

### Results on VAS

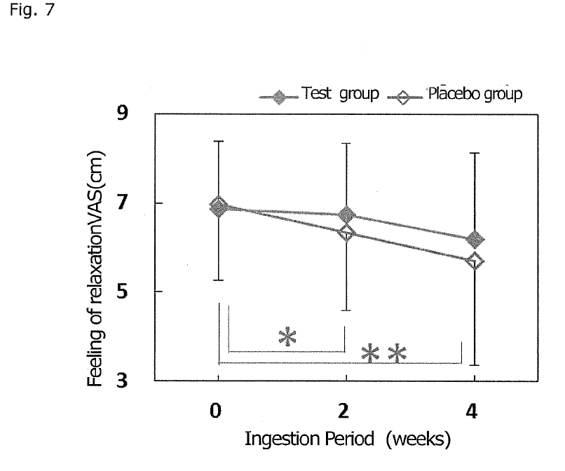

In the placebo group, the feeling of relaxation on VAS significantly decreased at two weeks after the onset of ingestion (p=0.005) and at four weeks after the onset of ingestion (p=0.011) compared to that on the starting date of ingestion (FIG. 7). In the test group, on the other hand, there was no difference in the feeling of relaxation from the starting date of the ingestion to four weeks after the onset of ingestion.

From these results, the test food was shown to suppress lessening of the feeling of relaxation.

### (5) Stress

### Results of SACL

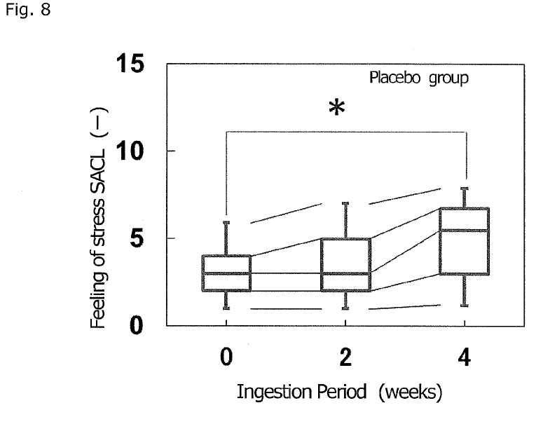

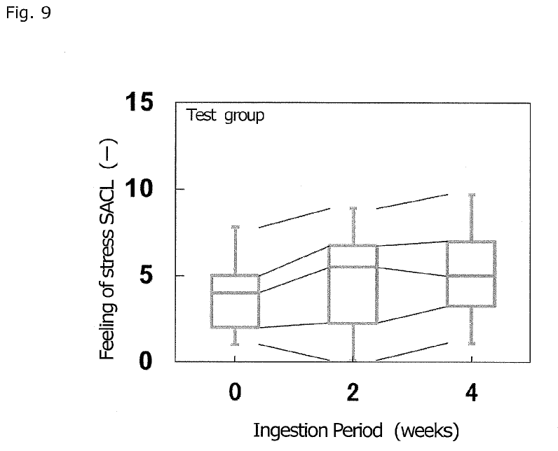

In the placebo group, the stress score at four weeks after the onset of ingestion significantly increased compared to that on the starting date of ingestion (p=0.012) (FIG. 8). In the test group, on the other hand, there was no difference in stress scores from the starting date of ingestion to four weeks after the onset of ingestion (FIG. 9).

From these results, the test food was shown to suppress increase of stress.

### (6) Ability to Concentrate

### Results on VAS

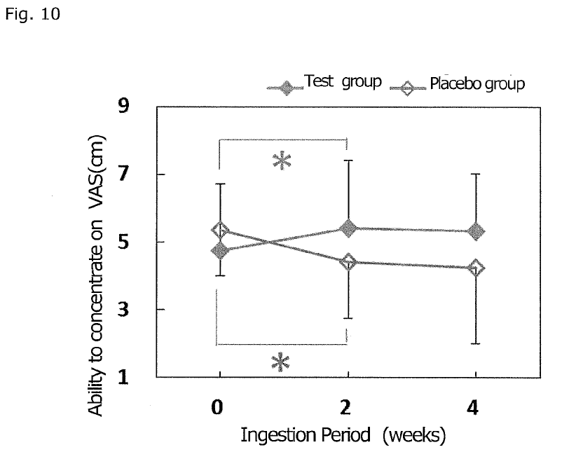

In the placebo group, ability to concentrate on VAS at two weeks after the onset of ingestion was significantly lower than that on the starting date of ingestion (p=0.025) (FIG. 10). In the test group, on the other hand, ability to concentrate on VAS at two weeks after the onset of ingestion was significantly higher than that on the starting date of ingestion (p=0.011). Also, ability to concentrate on VAS in the test group had a tendency to be higher than that in the placebo group at both two weeks after (p=0.082) and four weeks after (p=0.079) the onset of ingestion.

From these results, the test food was shown to improve ability to concentrate.

### Example 2: Ingestion Test 2

### [Design for Human Test]

An ingestion test was carried out by a randomized, double-blind placebo-controlled parallel-group trial. In addition, all the tests of the present application obtained an approval from the Ethical Review Committee at Kagawa Nutrition University and were performed under instructions by experts.

### [Age of Subjects, the Number of Subjects]

The ingestion test was conducted for a total of 52 healthy men and women subjects of 20 or more and less than 65 years of age.

### [Test Period]

A test was carried out for four weeks for a pre-observation period and eight weeks for the ingestion period, for a total of 12 weeks. The test period was set from Jan. 4 to Apr. 6, 2017, for a total of 12 weeks.

### [Measurement Items]

Questionnaire Evaluating Emotions

Using a questionnaire, the subjects were asked to address the following parameters.

### (1) VAS (Visual Analogue Scale):

A 10-cm-long line was used as a scale, at both ends on which were marked “feeling extremely” and “feeling not at all”. The evaluation was carried out by the position of markings put on the scale by the subjects to indicate the current state of their feeling with respect to the feeling of fatigue.

### (2) SACL (Stress/Wakefulness Check List):

The evaluation of emotions regarding stress or the feeling of wakefulness was performed by using a system of scoring from one to four to 30 questions.

Fatigue/Stress Measurement System

The heart electrical potential and pulse wave were measured and analyzed simultaneously by adhering a biosensor to the finger-tip of the subject. The balance of the autonomic nerves was measured using a “fatigue/stress measurement system” (Hitachi systems, Ltd.), which evaluates the functional state of the autonomic nerves.

The balance of the autonomic nerves was also evaluated by LF/HF. These are calculated from the integral values of intensity in power spectral density of heart rate variability in the areas of LF (low frequency) and HF (high frequency) components.

Evaluation of Fatigue in Everyday Life According to Self-Reported Diary

The time of awakening, sleep duration, the state of sleep, the degree of fatigue at the time of awakening (mental and physical), the degree of daily load (mental, physical and environmental), and the dietary degree of a day were recorded using a rating system of scoring from one to five by the subjects themselves. Alcohol consumption was recorded by scoring from one to three and the number of cigarettes the subjects smoked was also recorded.

### [Testing Schedule]

The subjects themselves wrote in their diary every day during the test period. After the pre-observation period of 4 weeks, subjects ingested 5 g of a test food or placebo food based on the result of grouping once a day for 8 weeks. Measurement dates during the test period were set on the starting date of the ingestion and 8 weeks after the onset of ingestion. One day before the measurement date, subjects themselves retrospectively filled out a questionnaire evaluating their emotions of the previous 7 days. Fatigue and stress were measured with a fatigue/stress measurement system for 150 seconds after resting for 2 minutes on the measurement date.

### [Grouping]

At the briefing session of the ingestion test, a body weight measurement and an interview about body height was carried out and BMI was calculated. Also, the measurement of the feeling of fatigue VAS was conducted. Grouping was randomly carried out based on sex, age, BMI and the feeling of fatigue VAS. There was no difference in data between the groups on the starting date of the ingestion. During the test period, a group ingesting the test food was set as the test group. A group ingesting the placebo food was set as the placebo group.

### [Statistical Analysis]

A parametric test was performed in respect of the feeling of fatigue VAS and a result was shown as a mean±standard error in a graph. A nonparametric test was performed in respect of SACL and the items recorded in the diaries. A result was shown as the average of the median values of each subject in a graph. The difference in the balance of the autonomic nerves between the starting date of the ingestion and 8 weeks after the onset of ingestion was calculated to get a ratio indicating how the balance of the autonomic nerves changed for each individual.

All the verification was conducted by a two-tailed test at a significance level of 5% (in the Figures, a significance level of 5% was expressed as “**”, and a significance level of 1% was expressed as “***” in comparison to the starting date of the ingestion or to pre-observation). In the case where p-values fall in the range of 0.05<p<0.1, the result was expressed as “*” and evaluated as “having a tendency”, although there was no significance. In addition, in comparison between the groups, a significance level of 5% was expressed as “##”, and a significance level of 1% was expressed as “###”. In the case where p-values fall in the range of 0.05<p<0.1, the result was expressed as “#” and evaluated as “having a tendency”, although there was no significance.

### [Results and Discussion]

### Feeling of Fatigue

### Results on VAS

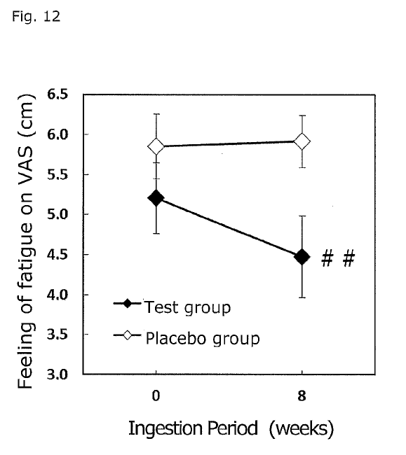

There was a significant difference in the feeling of fatigue on VAS between the groups 8 weeks after the onset of ingestion (FIG. 12). The feeling of fatigue of the subjects was unchanged in the placebo group, but the feeling of fatigue was alleviated in the test group.

### Results of the Feeling of Fatigue at the Time of Awakening According to the Diaries

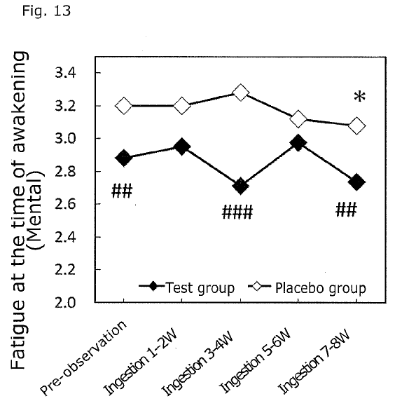

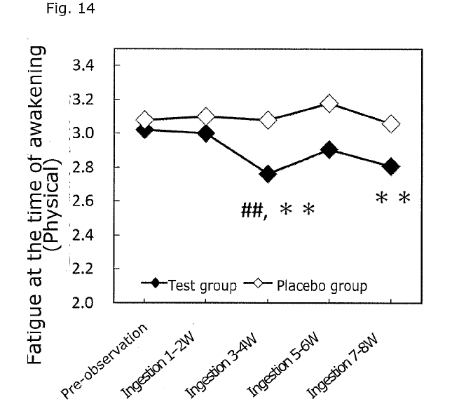

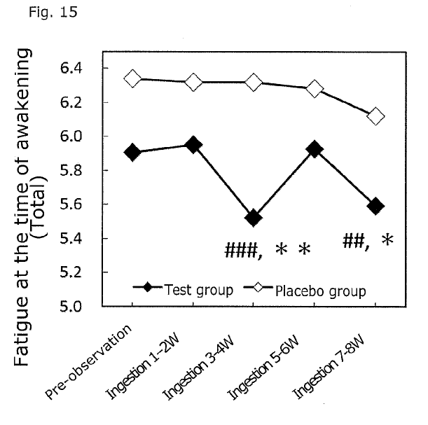

Diaries filled out by subjects every day were gathered once every two weeks. There was no change in the living conditions concerning fatigue such as the time of awakening, sleep duration, degree of daily load (mental, physical and environmental), dietary degree of a day, alcohol consumption, and number of cigarettes smoked. Therefore, in the placebo group, there was no change in the feeling of fatigue at the time of awakening, mental fatigue, physical fatigue and total fatigue (FIGS. 13, 14, 15). However, in the test group, the feeling of fatigue at the time of awakening, mental fatigue, physical fatigue and total fatigue, was alleviated during the ingestion period. Thus, there was a significant difference in comparison to the placebo group, and to pre-observation.

### (Feeling of Sound Sleep)

### Results of the Feeling of Sound Sleep According to the Diaries

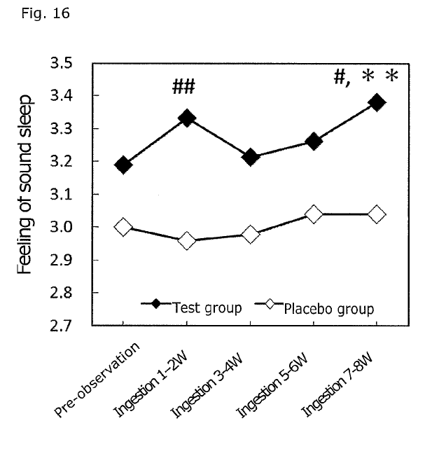

In the placebo group, there was no change in the feeling of sound sleep during the test period. However, in the test group, the feeling of sound sleep was improved during the ingestion period, and there was a significant difference in comparison to the placebo group and to pre-observation (FIG. 16).

### Feeling of Wakefulness

### Results of the Feeling of Wakefulness According to SACL

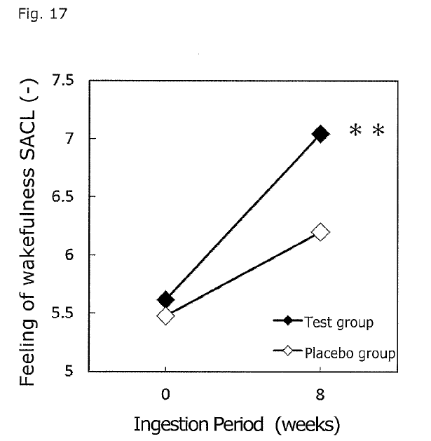

Regarding the feeling of wakefulness, there was no significant result by the ingestion in the placebo group. However, in the test group, the feeling of wakefulness was significantly increased as compared to the starting of the ingestion (FIG. 17).

### (Balance of the Autonomic Nerves)

### Results According to Fatigue/Stress Measurement System

The balance of the autonomic nerves is expressed as the ratio of LF/HF. The state in which LF/HF ratio is smaller than a reference value indicates the state in which activity of the parasympathetic nervous system is high and that of the sympathetic nervous system is low. For the subjects whose LF/HF ratio was lower than the reference value at the starting date of the ingestion, it is desirable to raise this value within a range of the reference value. Therefore, for those who showed that the difference between the starting date of the ingestion and 8 weeks after the onset of ingestion was lower than the reference value, when the difference increased within a range not exceeding the upper limit of the reference value, they were regarded as in a state of “improvement”. When the difference decreased or increased beyond the upper limit of the reference value, they were regarded as in a state of “exacerbation”.

On the other hand, the state in which the ratio of LF/HF is high indicates that activity of the sympathetic nervous system is high and that of the parasympathetic nervous system is low. For the subjects whose LF/HF ratio was equal to or larger than the reference value at the starting date of the ingestion, it is desirable to lower this value within a range of the reference value. Therefore, for those who showed that the difference between the starting date of the ingestion and 8 weeks after the onset of ingestion was equal to or larger than the reference value, when the difference decreased within a range not falling below the lower limit of the reference value were regarded as in a state of “improvement”. When the difference increased or decreased below the lower limit of the reference value, they were regarded as in a state of “exacerbation”.

For the subjects who showed that the difference in the ratio of LF/HF on the starting date of the ingestion was within the reference value, when the ratio was within a range of the reference value eight weeks after the onset of ingestion, the subjects were regarded as in a state of “change within the reference value”. When the ratio deviated from the range of the reference value, they were regarded as in a state of “exacerbation”.

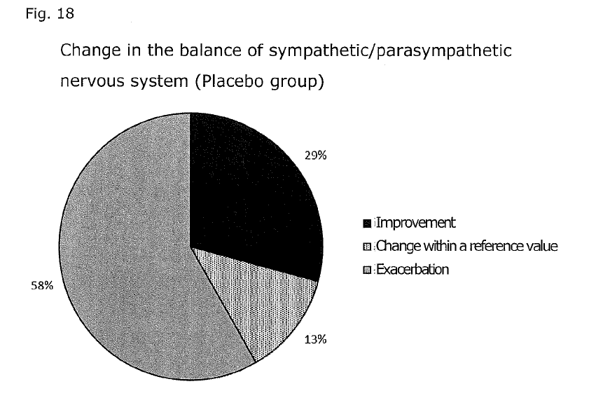

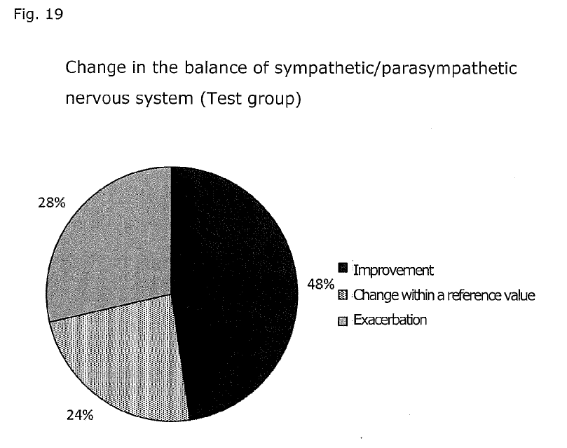

As a result, in the placebo group, the proportion of subjects who were in “exacerbation” was high at 58%. It was thought that the balance of the autonomic nerves was prone to be disturbed because the period when the test was carried out was during the change of seasons (FIG. 18). On the other hand, the proportion of subjects who were in “exacerbation” was suppressed low at 28% in the test group (FIG. 19). Also, while the proportion of the subjects who were in “change within the reference value” was 13% in the placebo group, it was high at 24% in the test group. It was found that ingesting the test food enabled subjects to maintain a balanced state of their autonomic nerves. Furthermore, while the proportion of the subjects who were in “improvement” state was 29% in the placebo group, it was 48% in the test group. It was found that ingesting the test food enabled subjects to improve the balance of their autonomic nerves.

As described above, the alleviation effect of fatigue was recognized by an anti-fatigue food composition according to the present invention. In this respect, it was found that the feeling of sound sleep was improved while the feeling of wakefulness was elevated, and the feeling of fatigue at the time of awakening was alleviated mentally and physically. The effect of improving the feeling of sound sleep while the feeling of wakefulness is elevated is thought to be obtained by maintaining the balanced state of the autonomic nerves and improving the balance of autonomic nerves.

### Example 3: A Comparison of Ingredients in Aged Garlic Extracts Produced by Various Methods

A comparison of the amounts of ingredients was performed among “aged garlic extract” produced by a method described in Japanese Unexamined Patent Application Publication No. 2014-45693 by the present inventors, a garlic extract aged by alcohol extraction (Sample A), and a garlic extract aged by a heating/humidity conditioning method (Sample B).

Samples other than the “aged garlic extract”, namely (Sample A and B) were commercially available products whose ingredients were analyzed. Sample A contains other raw materials as well as garlic. Therefore, only ingredients derived from garlic in each sample were analyzed and the comparison was performed in terms of the proportion of these ingredients.

### Analysis Method

### Measurement Method for Allicin

Among the ingredients derived from the garlic contained in Sample A, the concentration of allicin was measured as follows. First, to 10 g of a sample is added 25 mL of a 45% methanol solution, the mixture is stirred well and filled up to a constant volume of 50 mL (by further adding the solution). After this diluted solution is subjected to centrifugation and a supernatant is recovered, the diluted solution is further filtered by ultrafiltration (Merck Millipore, Amicon Ultra-0.5, molecular weight cut off 3,000) to recover a low molecular weight fraction in the sample. The recovered sample with low molecular weight can be analyzed by liquid chromatography. Preferably, the analysis can be conducted by HPLC, and HPLC conditions can be set as follows.

### (HPLC Analysis Conditions)

Device: LC-20A system (SHIMADZU CORPORATION)
Column: Symmetry C18 (5 μm, 4.6 mm×150 mm)
Mobile phase: Methanol/Mill-Q water=45/65 (isocratic mode)
Column oven temperature: room temperature
Flow rate: 1.0 mL/min
Detection wavelength: 210 nm

### Measurement Method for Sulfides (Allyl Methyl Sulfide, Dimethyl Disulfide, Dimethyl Trisulfide, Allyl Sulfide, Diallyl Disulfide, and Diallyl Trisulfide)

Among the ingredients derived from the garlic contained in Sample A, the concentration of sulfides was measured as follows. First, to 10 g of sample was added 30 g of distilled water and the mixture was stirred well. To the mixture was further added 40 mL of hexane and the resultant mixture was shaken for more than one minute. This mixture was subjected to centrifugation and a hexane layer in the supernatant is recovered. After hexane was removed from the recovered supernatant by vacuum concentration, the residuewas dissolved in acetone and can be analyzed by liquid chromatography. Preferably, the analysis can be conducted by HPLC (High Performance Liquid Chromatography), and HPLC conditions can be set as follows.

### (HPLC Analysis Conditions)

Device: LC-20A system (SHIMADZU CORPORATION)
Column: Symmetry C18 (5 μm, 4.6 mm×150 mm)
Mobile phase solution A: Acetonitrile/Milli-Q water/Tetrahydrofuran=40/57/3
Mobile phase liquid B: Acetonitrile/Milli-Q water/Tetrahydrofuran=70/27/3
Gradient: Set as in the following Table 5.
Column oven temperature: 40° C.
Sample chamber temperature: Room temperature
Flow rate: 1.0 mL/min
Detection wavelength: 210 nm

TABLE 5

| | Initial | | | | | | |
|---|---|---|---|---|---|---|---|
| Time (min.) | value | 20.0 | 30.0 | 40.0 | 45.0 | 46.0 | 53.0 |
| Solution A (%) | 100.0 | 100.0 | 40.0 | 40.0 | 0.0 | 100.0 | 100.0 |
| Solution B (%) | 0.0 | 0.0 | 60.0 | 60.0 | 100.0 | 0.0 | 0.0 |

### Measurement Method for S-Allyl Cysteine, S-Methylcysteine, Alliin, Isoalliin, and Methiin

Among the ingredients derived from the garlic contained in sample A, the concentration of S-allyl cysteine, S-methylcysteine, alliin, isoalliin, and methiin can be measured, for example, in a method as follows. First, to 10 g of a sample is added 490 g of distilled water and the mixture is stirred well. This diluted solution can be analyzed by liquid chromatography using AccQ-Tag amino acid analysis (Waters). Preferably, the analysis can be carried out by UPLC (registered trademark) (Ultra High Performance Liquid Chromatography) and the UPLC conditions can be as follows.

### (UPLC Analysis Conditions)

Device: UPLC system (Nihon Waters K. K.)
Column: AccQ-Tag Ultra C18 (1.7 μm, 2.1 mm×100 mm)
Mobile phase solution A: AccQ-Tag Ultra eluant A concentrate

### (Waters)

Mobile phase solution B: Solution obtained by diluting solution D with Milli-Q water by a factor of 10
Mobile phase solution C: Milli-Q water
Mobile phase solution D: Acetonitrile/formic acid=97/3
Gradient: Set as in the following Table 6.
Column oven temperature: 43° C.
Sample chamber temperature: 20° C.
Flow rate: 0.7 mL/min
Detection wavelength: Fluorescence Ex (excitation wavelength) 266 nm, Em (fluorescence wavelength) 473 nm

TABLE 6

| Time | Initial | | | | | | | | | |
|---|---|---|---|---|---|---|---|---|---|---|
| (min.) | value | 0.29 | 5.49 | 7.1 | 7.3 | 7.69 | 7.99 | 8.59 | 8.68 | 10.20 |
| Solution | 10.0 | 9.9 | 9.0 | 8.0 | 8.0 | 7.8 | 4.0 | 4.0 | 10.0 | 10.0 |
| A (%) | | | | | | | | | | |
| Solution | 0.0 | 0.0 | 80.0 | 15.6 | 15.6 | 0.0 | 0.0 | 0.0 | 0.0 | 0.0 |
| B (%) | | | | | | | | | | |
| Solution | 90.0 | 90.1 | 11.0 | 57.9 | 57.9 | 70.9 | 36.3 | 36.3 | 90.0 | 90.0 |
| C (%) | | | | | | | | | | |
| Solution | 0.0 | 0.0 | 0.0 | 18.5 | 18.5 | 21.3 | 59.7 | 59.7 | 0.0 | 0.0 |
| D (%) | | | | | | | | | | |

### Measurement Method for Cycloalliin

Among the ingredients derived from the garlic contained in Sample A, the concentration of cycloalliin can be measured, for example, in a method as follows. First, to 10 g of a sample is added 25 mL of a 45% methanol solution, the mixture is stirred well and filled up to a constant volume of 50 mL. This diluted solution is subjected to centrifugation and a supernatant is recovered. The recovered sample can be analyzed by liquid chromatography. Preferably, the analysis can be conducted by HPLC, and HPLC conditions can be set as follows.

### (HPLC Analysis Conditions)

Device: LC-20A system (SHIMADZU CORPORATION)
Column: Asahipak NH2P-50 4E (5 μm, 4.6 mm×250 mm)
Mobile phase: Phosphoric acid/Milli-Q water/Acetonitrile=0.1/20/80 (isocratic mode)
Column oven temperature: 40° C.
Sample chamber temperature: Room temperature
Flow rate: 1.0 mL/min
Detection wavelength: 210 nm

### [Results of a Comparative Test]

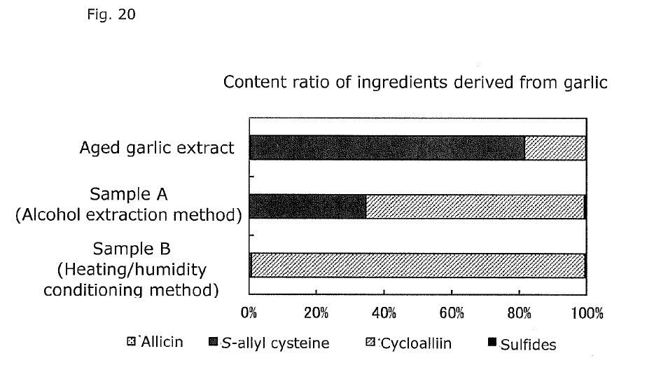

The proportions of the measured ingredients derived from the garlic are shown in FIG. 20. The contents of allicin and sulfides were small in any sample, and S-methylcysteine, alliin, isoalliin, and methiin were not contained.

On the other hand, the proportions of S-allyl cysteine and cycloalliin in each sample differed considerably. S-allyl cysteine accounted for approximately 80% (of the ingredients) in the aged garlic extract prepared by a method described in Japanese Unexamined Patent Application Publication No. 2014-45693, while cycloalliin mostly accounted for the ingredients in Sample B. In Sample A, approximately 35% of the ingredients was S-allyl cysteine and approximately 65% was cycloalliin, which was intermediate between other two garlic extracts.

As described above, the contents in the aged garlic extract prepared by the method described in Japanese Unexamined Patent Application Publication No. 2014-45693, and two kinds of samples in which garlic was similarly aged were analyzed. It was found that the component composition in each sample varied according to the method of aging.

### SUMMARY

As mentioned above, the anti-fatigue food composition according to the present invention has been found to possess effects such as suppression of accumulation of the feeling of fatigue in everyday life, improvement in work efficiency, suppression of lessening of the feeling of relaxation, suppression of accumulation of the feeling of stress, improvement in ability to concentrate, suppression of lessening of the feeling of wakefulness, improvement in the feeling of wakefulness, suppression of lessening of the state of liveliness, improvement in the feeling of sound sleep, alleviation of the feeling of fatigue at the time of awakening, maintenance of the balance of the autonomic nerves and improvement in the balance of the autonomic nerves.

A major factor of fatigue is that the autonomic nerve center in the brain gets fatigued, and a direct cause of fatigue is oxidative stress due to active oxygen species. Various antioxidant substances such as vitamins, polyphenols and carotenoids other than S-allylcysteine are known to alleviate oxidative stress, but only a part of antioxidant substances show anti-fatigue effects in vivo because their mechanisms of anti-oxidation vary and their antioxidative actions are exerted at different time points in vivo. Especially, substances that can directly contribute to the recovery from fatigue in the brain are limited to only a few substances among them such as S-allylcysteine can pass through the blood-brain barrier. Therefore, the well-aged garlic paste blended with the aged garlic extract aged by heating in the present invention containing abundant S-allylcysteine is thought to be useful with respect to its effective suppression of the feeling of fatigue.

Also, the feeling of fatigue is a biological alarm indicating fatigue in the brain. Therefore, suppression of the feeling of fatigue alone has nothing left except to nullify the biological alarm, and does not seem to lead to improved subsequent performances. However, the paste according to the present application not only suppresses accumulation of the feeling of fatigue but also has effect of suppression of lessening of the feeling of relaxation and suppression of increase of the feeling of stress, resulting in the improvement in ability to concentrate and the increase of efficiency in simple work. For these reasons, the present invention can be said to be a more excellent anti-fatigue agent exerting the effect of suppression of accumulation of the feeling of fatigue and at the same time the effect of improvement in performance.

Further, the component composition of the garlic extract aged by heating according to the present invention is significantly different from the component compositions of the garlic extract aged by alcohol extraction, and the garlic extract aged by a heating/humidity conditioning method.

### INDUSTRIAL APPLICABILITY

From these results, an aged garlic paste prepared by blending an aged garlic extract, containing abundant high functional ingredients such as S-allylcysteine, with other food materials was found to suppress accumulation of the feeling of fatigue over days, and at the same time to improve the efficiency of simple work (performance). Further, it was found that the aged garlic paste according to the present invention can maintain or improve the balance of the autonomic nerves, thereby improve in the feeling of wakefulness and the feeling of sound sleep, and suppress the feeling of mental and physical fatigue at the time of awakening. The anti-fatigue food composition of the present invention is useful in the field of health food and pharmaceutical products.

1. An anti-fatigue agent containing an aged garlic extract aged by heating.

2. The anti-fatigue agent according to claim 1, wherein the anti-fatigue agent is used for suppressing of accumulation of the feeling of fatigue in daily life.

3. The anti-fatigue agent according to claim 1, wherein the anti-fatigue agent is used for obtaining at least one efficacy selected from the group consisting of the followings;
(1) efficacy of improving in working efficiency;
(2) efficacy of suppressing of lessening of the feeling of relaxation;
(3) efficacy of suppressing of accumulation of the feeling of stress;
(4) efficacy of improving in ability to concentrate;
(5) efficacy of suppressing of lessening of feeling of wakefulness;
(6) efficacy of improving of feeling of wakefulness;
(7) efficacy of suppressing of lessening of the state of liveliness;
(8) efficacy of improving in the feeling of sound sleep;
(9) efficacy of alleviating the feeling of fatigue at the time of awakening;
(10) efficacy of maintaining of balance of the autonomic nerves;
(11) efficacy of improving in the balance of the autonomic nerves.

4. An anti-fatigue food containing the anti-fatigue agent according to claim 1.

5. The anti-fatigue food according to claim 4, wherein the food is a past containing an aged garlic extract aged by heating, brown sugar, black vinegar, and sugar.

6. An anti-fatigue composition containing an aged garlic extract aged by heating.

7. The anti-fatigue composition according to claim 6, wherein the composition is used for suppressing of accumulation of the feeling of fatigue in daily life.

8. The anti-fatigue composition according to claim 7, wherein the composition is used for obtaining at least one of efficacy selected from the group consisting of the followings;
(1) efficacy of improving in working efficiency;
(2) efficacy of suppressing of lessening of the feeling of relaxation;
(3) efficacy of suppressing of accumulation of the feeling of stress;
(4) efficacy of improving in ability to concentrate;
(5) efficacy of suppressing of lessening of feeling of wakefulness;
(6) efficacy of improving of feeling of wakefulness;
(7) efficacy of suppressing of lessening of the state of liveliness;
(8) efficacy of improving in the feeling of sound sleep;
(9) efficacy of alleviating the feeling of fatigue at the time of awakening;
(10) efficacy of maintaining of balance of the autonomic nerves;
(11) efficacy of improving in the balance of the autonomic nerves.

9. The anti-fatigue composition according to claim 7, wherein the composition is a food composition.

10. The anti-fatigue composition according to claim 9, wherein the composition is a paste containing an aged garlic extract aged by heating, brown sugar, black vinegar, and sugar.

11. An anti-fatigue food containing the anti-fatigue agent according to claim 2.

12. An anti-fatigue food containing the anti-fatigue agent according to claim 3.

13. The anti-fatigue food according to claim 11, wherein the food is a past containing an aged garlic extract aged by heating, brown sugar, black vinegar, and sugar.

14. The anti-fatigue food according to claim 12, wherein the food is a past containing an aged garlic extract aged by heating, brown sugar, black vinegar, and sugar.

15. The anti-fatigue composition according to claim 8, wherein the composition is a food composition.

16. The anti-fatigue composition according to claim 15, wherein the composition is a paste containing an aged garlic extract aged by heating, brown sugar, black vinegar, and sugar.
